(19) United States
(12) Patent Application Publication
(10) Pub. No.: US 2004/0133147 A1
(43) Pub. Date: Jul. 8, 2004

(54) Intestinal bypass device to treat obesity

(76) Inventor: Sang Hoon Woo, Durham, NC (US)

Correspondence Address: Sang Hoon Woo, MD, 5208A Penrith Drive, Durham, NC, 27713, US

(21) Appl. No.: 10/694,149

(22) Filed: Oct. 27, 2003

Related U.S. Application Data
(60) Provisional application No. 60/424,248, filed on Nov. 6, 2002.

(51) Int. Cl. A61F 2/04

(52) U.S. Cl. 604/9; 623/23.68; 128/898

(57) ABSTRACT

The present invention provides a device for causing weight loss in obese patients comprising an implant that creates an intestinal bypass between a first region of intestine and a second region of intestine. In one embodiment, the implant comprises an adjustable opening to adjust the fraction of food material passing through the intestinal bypass. Also disclosed is a method for causing weight loss in obese patients comprising the steps of surgically creating an intestinal bypass with an adjustable opening, calculating an expected weight loss and an expected electrolyte balance in the patient, periodically monitoring the patient's weight loss and electrolyte balance and adjusting the size of the adjustable opening if necessary.

This application claims priority to U.S. Provisional Application No. 60/424,248 filed Nov. 6, 2002.

### BACKGROUND OF THE INVENTION

The present invention relates to surgical devices to treat obesity. More particularly, the present invention relates to surgical implants for causing weight loss.

Obesity is a serious health problem especially in developed countries. Approximately 60 million adults in the U.S. are obese. Obesity leads to several health problems such as increase of risk of illness and death due to coronary artery disease, diabetes, stroke, hypertension, and kidney and gallbladder disorders and some types of cancer. It also increases the risk of developing osteoarthritis and a condition called sleep apnea defined as periodic cessation of breathing during sleep (Source: Medline). Obesity also causes several psychosocial problems like depression and loss of self-esteem.

Obesity has high medical costs due to the high prevalence of obesity and the various health problems associated with it. In a study conducted in 1998, the direct medical costs due to obesity were estimated to be $51.64 billion in the US (Source: Website of the American Obesity Association). These costs could increase in the future as the prevalence of obesity is steadily increasing. In the United States, the percentage of children and adolescents who are obese has doubled in the last 20 years. Thus, there is an urgent need to treat this serious health problem.

Obesity is treated by reducing the patient's weight. Although several methods are available to achieve weight loss, none of them have been entirely successful in causing the desired weight loss. Weight loss methods can be broadly divided into diet modification, exercise therapy, pharmacological therapy and surgical procedures. The most common method of weight loss is diet modification. The aim of diet modification techniques is to reduce the number of calories that are consumed by the patient. However, the success of a weight loss program based on diet modification critically depends upon the patient strictly following the prescribed diet.

Exercise therapy causes weight loss through aerobic exercises. Like diet modification methods, the success of a weight loss program based on exercise therapy critically depends upon the patient regularly performing the prescribed exercises.

Pharmacological therapy uses specific medications that cause weight loss. However, the use of weight loss medications causes side effects. Further, when the weight loss medications are discontinued, the lost weight is regained.

Surgical procedures are used for weight loss when diet modification, exercise therapy and pharmacological therapy fail to cause required weight loss. The most commonly used surgical procedures for weight loss are Roux-en-Y gastric bypass procedure, restrictive gastric operations, malabsorptive operations such as biliopancreatic diversion and intestinal bypass procedure. The Roux-en-Y gastric bypass procedure involves creating a stomach pouch out of a small portion of the stomach and attaching it directly to the small intestine, bypassing a large part of the stomach and duodenum. The small stomach pouch holds much smaller amounts of food at a time, and hence the patient experiences a feeling of satiety even after eating a small quantity of food. Also, fat absorption from food is substantially reduced as the food bypasses a large portion of the duodenum.

Restrictive gastric operations cause weight loss by restricting the food intake by the patient. A portion of the stomach is surgically modified to form a small pouch. The food enters the pouch from the esophagus. The outlet from the pouch to the rest of the stomach is restricted. This restriction delays the emptying of food from the pouch, causing a feeling of fullness even after consuming small amounts of food.

Malabsorptive operations such as biliopancreatic diversion cause weight loss by restricting the food intake and also by reducing the fraction of calories absorbed by the body from the digested food. In a biliopancreatic diversion, portions of the stomach are removed along with the duodenum and the jejunum. This reduces the fraction of calories absorbed from the digested food, thereby causing weight loss.

Conventional intestinal bypass procedures cause weight loss by removing a portion of the small intestine and reconnecting the remaining portion of the small intestine. Removal of a portion of the small intestine reduces the effective length of the small intestine. This reduces the amount of nutrients that are absorbed by the body from the food and causes weight loss. It is also associated with severe side effects.

The abovementioned surgical procedures are highly invasive and require major modifications to the patient's anatomy. Further, the anatomical modifications due to these procedures cannot be frequently adjusted to adjust the rate of weight loss. Also, if these surgical procedures cause severe side effects to the patient, the anatomical modifications cannot be reversed easily.

There are several surgical procedures for causing weight loss that use implants like intragastric balloons and vagus nerve stimulation devices. Intragastric balloons cause weight loss by occupying a significant portion of the stomach lumen and inducing a feeling of satiety in the patient. However, the intragastric balloons cannot be easily adjusted on a regular basis to adjust the rate of weight loss. Vagus nerve stimulation devices stimulate the vagus nerve of a patient by electrical currents to produce a sensation of satiety. Vagus nerve stimulation devices face the problems of accidental stimulation and potential of harm to the patient in the presence of strong electromagnetic fields. Also they have been associated with unpleasant side effects.

Thus, there is a need for an obesity treatment that does not need significant modifications to the patient's anatomy. Further, there is a need for an obesity treatment whose parameters can be adjusted frequently to adjust the rate of weight loss. Further, there is a need for an obesity treatment whose parameters can be adjusted with minimal discomfort to the patient. Further, there is a need for an obesity treatment that can be easily reversed if the patient experiences significant side effects.

### BRIEF SUMMARY OF THE INVENTION

An object of the present invention is to provide an obesity treatment that does not cause significant modifications to the patient's anatomy as compared to other surgical treatments. Another object of the present invention is to provide an obesity treatment whose parameters can be adjusted frequently to adjust the rate of weight loss. Another object of the present invention is to provide an obesity treatment whose parameters can be adjusted with minimal discomfort to the patient.

To achieve the foregoing objects, and in accordance with the purpose of the present invention, the present invention provides a device for causing weight loss in obese patients comprising an implant that creates an intestinal bypass between a first region of intestine and a second region of intestine. A part of food material passing through the intestine from the first region of intestine to the second region of intestine is diverted through the intestinal bypass. As the intestine is the main site for absorption of nutrients from food material, diversion of a part of food material through the bypass graft causes a reduction in the total nutrients absorbed by the body from the food material. This causes the patient to lose weight. In one embodiment, the implant comprises an adjustable opening to adjust the fraction of food material passing through the intestinal bypass and hence adjust the rate of weight loss.

The present invention also provides a method for causing weight loss in obese patients comprising the steps of surgically creating an intestinal bypass with an adjustable opening, calculating an expected weight loss and an expected electrolyte balance in the patient, periodically monitoring the patient's weight loss and electrolyte balance and adjusting the size of the adjustable opening if necessary.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 illustrates the general working environment of the invention;

FIG. 2 illustrates an embodiment of the invention;

FIG. 3 illustrates a second embodiment of the invention;

FIG. 4 illustrates a third embodiment of the invention;

FIG. 5 illustrates a sectional view of an embodiment of the invention;

FIG. 6 illustrates an embodiment of the adjustable opening of the invention;

FIG. 7 illustrates a second embodiment of the adjustable opening of the invention; and

FIG. 8 illustrates the method of the present invention to achieve weight loss in obese patients.

### DETAILED DESCRIPTION OF THE INVENTION

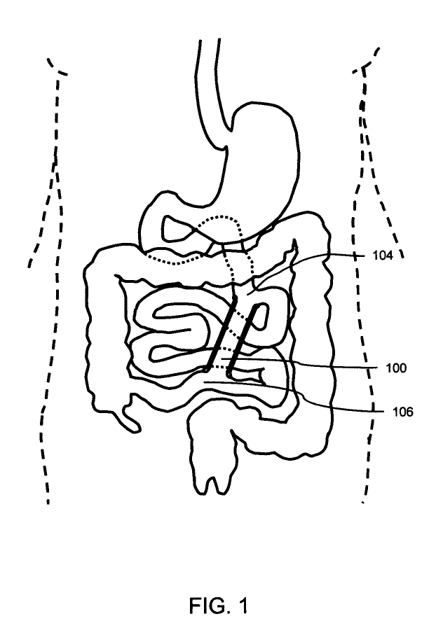

FIG. 1 illustrates the general working environment of the invention. The invention comprises an implant 100 that connects a first region 104 of the intestine to a second region 106 of the intestine to create an intestinal bypass. First region 104 is located on the small intestine. Second region 106 is located downstream from first region 104. Second region 106 can be located on the small intestine or the large intestine.

The invention achieves weight loss by reducing the amount of food material that is absorbed by the body. A portion of food material passing through the intestine from the first region 104 to the second region 106 is diverted through the intestinal bypass. The portion of food material passing through the intestinal bypass is unabsorbed. As the intestine is the main site for absorption of the food material, diversion of a portion of food material through the intestinal bypass reduces the net food material absorbed by the body. This causes the patient to lose weight.

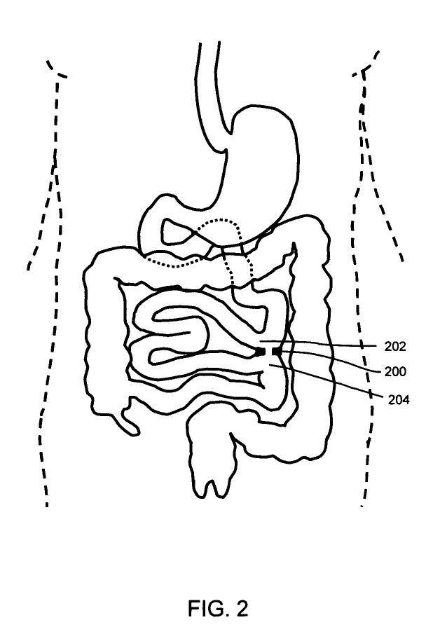

FIG. 2 illustrates an embodiment of the invention. The invention comprises an implant 200 that comprises a ring that directly connects a first region 202 of the intestine to a second region 204 of the intestine to create an intestinal bypass.

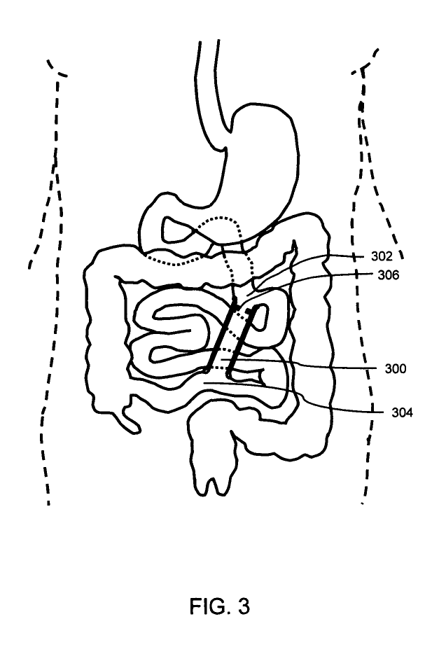

FIG. 3 illustrates a second embodiment of the invention. The invention comprises a tubular implant 300 that connects a first region 302 of the intestine to a second region 304 of the intestine to create an intestinal bypass. Tubular implant 300 comprises an adjustable opening 306 to adjust the rate of weight loss.

The rate of weight loss can be controlled by adjusting the size of adjustable opening 306. A larger opening will cause a greater portion of the food material to pass through the intestinal bypass. This will reduce the amount of nutrients absorbed by the intestine from the food material and thus increase the rate of weight loss. Similarly, reducing the size of adjustable opening 306 will reduce the rate of weight loss.

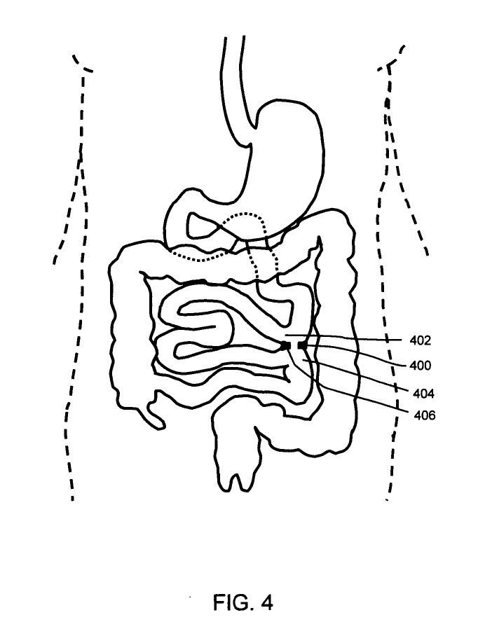

FIG. 4 illustrates a third embodiment of the invention. The invention comprises an implant 400 that comprises a ring that directly connects a first region 402 of the intestine to a second region 404 of the intestine to create an intestinal bypass. Implant 400 comprises an adjustable opening 406 to adjust the rate of weight loss.

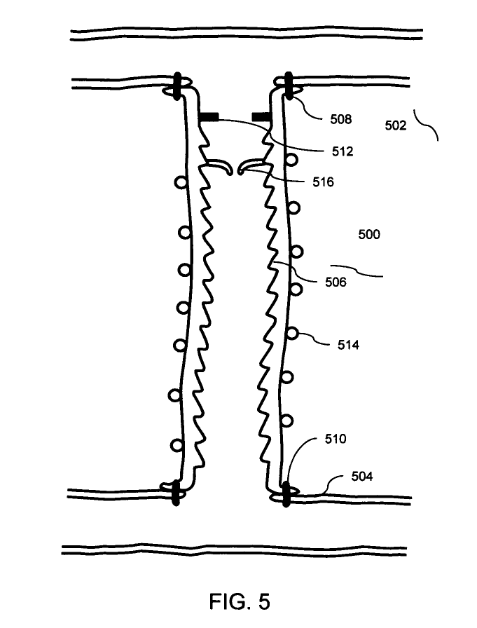

FIG. 5 illustrates a sectional view of an embodiment of the invention. An intestinal bypass graft 500 is used to create a bypass between a first region 502 of the intestine and a second region 504 of the intestine. Intestinal bypass graft 500 comprises a tubular implant 506. Tubular implant 506 can be made of suitable biocompatible materials like silicone gel, polyurethane, ultra high molecular weight polyethylene, polyethylene terephthalate, polypropylene, polytetrafluoroethylene and polyamides. In one embodiment, the walls of the tubular implant are hollow and are filled with a filler material. Examples of filler material that can be used are silicon gel, saline, soybean oil, hydro gel, polyvinylprolidone, polyethylene glycol, and hyaluronic acid. The inner surface of tubular implant 506 has a series of projections. The projections help the flow of food material in the intestine in a single direction. One end of tubular implant 506 is connected to first region 502 of intestine by one or more fasteners 508 to create an end-to-side anastomosis. Fasteners 508 are biocompatible. Examples of materials that can be used as fasteners 508 are sutures, clips, staples, screws, tags and adhesives. The other end of tubular implant 506 is connected to second region 504 of intestine by one or more fasteners 510 to create an end-to-side anastomosis. Fasteners 510 are biocompatible. Examples of materials that can be used as fasteners 510 are sutures, clips, staples, screws, tags and adhesives. Tubular implant 506 is provided with an adjustable opening 512. Adjustable opening 512 regulates the amount of food that passes through intestinal bypass graft 500. Increasing the size of adjustable opening 512 increases the amount of food passing through intestinal bypass graft 500. This reduces the amount of consumed food that is absorbed by the patient's body and increases the rate of weight loss. Similarly, reducing the size of adjustable opening 512 reduces the rate of weight loss. Thus the rate of weight loss can be regulated by changing the size of adjustable opening 512. Tubular implant 506 is further provided with an elastic mechanism 514. Elastic mechanism 514 provides elasticity to intestinal bypass graft 500. The motion of the patient and the peristaltic motion of the patient's intestines cause various regions of intestinal bypass graft 500 to move with respect to each other. This movement facilitates the flow of food material passing through intestinal bypass graft 500. In one embodiment, elastic mechanism 514 is in the form of a spring wound around tubular implant 506. Several biocompatible materials like titanium alloys, stainless steel alloys or elastic biocompatible polymers can be used for constructing the spring. Tubular implant 506 further comprises a valve 516. Valve 516 allows the flow of food material only in a single direction and thus prevents backflow of the food material. Valve 516 can be a mechanical valve or a bioprosthetic valve. Examples of mechanical valves that can be used are ball valves, single-leaflet (tilting disk) valves and bileaflet valves. They can be made of one or more biocompatible materials like collagen, stainless steel, titanium, pyrolytic carbon, Teflon or Dacron. Bioprosthetic valves can be made from animal or human tissues.

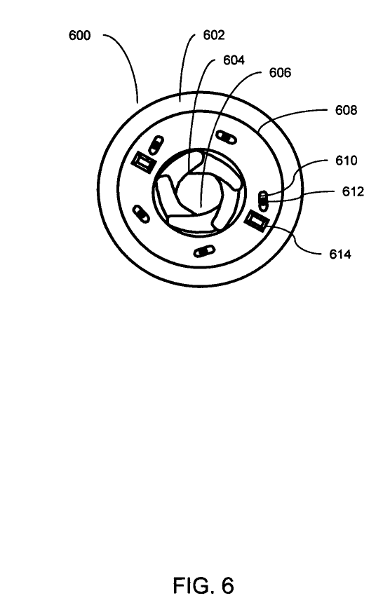

FIG. 6 illustrates an embodiment of the adjustable opening of the invention. The adjustable opening comprises an iris diaphragm 600. Iris diaphragm 600 comprises a base plate 602. Base plate 602 is annular in shape. Iris diaphragm 600 further comprises a plurality of blades 604. Each blade is attached to base plate 602 by a pivot in such a way that blades 604 enclose a lumen 606. Iris diaphragm 600 further comprises a blade actuating ring 608 attached coaxially to base plate 602. Blade actuating ring 608 can rotate around its axis. Blade actuating ring 608 is provided with a plurality of slots 610. The number of slots on blade actuating ring 608 is equal to the number of blades attached to base plate 602. Each blade is provided with a projection 612. Projection 612 of each blade slides within a slot on blade actuating ring 608. Thus, each blade is pivoted on base plate 602 and communicates with blade actuating ring 608. Blade actuating ring 608 is further provided with a plurality of gripping slots 614. Gripping slots 614 are used in gripping and rotating blade actuating ring 608. Rotation of blade actuating ring 608 changes the orientation of blades 604. This changes the size of lumen 606. Thus, the size of adjustable opening in the invention can be changed by rotating blade actuating ring 608. In one embodiment, blade actuating ring 608 is rotated using endoscopic means. Several biocompatible materials like titanium alloys, stainless steel alloys or elastic biocompatible polymers can be used for constructing the iris diaphragm 600.

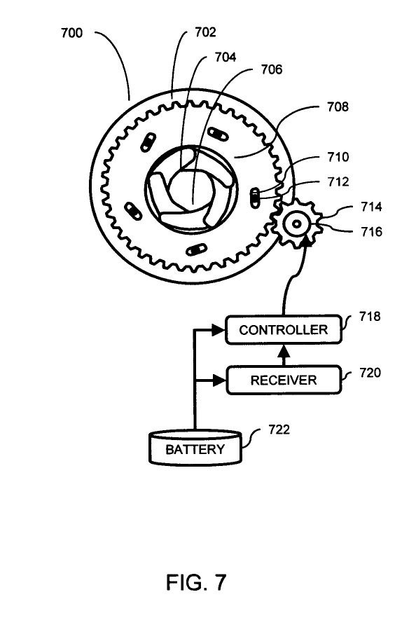

FIG. 7 illustrates a second embodiment of the adjustable opening of the invention. The size of the adjustable opening is controlled using electromagnetic signals. The adjustable opening comprises an iris diaphragm 700. Iris diaphragm 700 comprises a base plate 702. Base plate 702 is annular in shape. Iris diaphragm 700 further comprises a plurality of blades 704. Each blade is attached to base plate 702 by a pivot in such a way that blades 704 enclose a lumen 706. Iris diaphragm 700 further comprises a blade actuating ring 708 attached coaxially to base plate 702. Blade actuating ring 708 can rotate around its axis and can act a gear. Blade actuating ring 708 is provided with a plurality of slots 710. The number of slots on blade actuating ring 708 is equal to the number of blades attached to base plate 702. Each blade is provided with a projection 712. Projection 712 of each blade slides within a slot on blade actuating ring 708. Thus, each blade is pivoted on base plate 702 and communicates with blade actuating ring 708. The outer diameter of blade actuating ring 708 is geared to a driver gear 714. Driver gear 714 is connected to a control mechanism comprising a motor 716 and a controller 718 that supplies electric current to motor 716. Controller 718 is connected to a receiver 720. Receiver 720 receives electromagnetic signals and converts the received electromagnetic signals to electric signals and transmits the electric signals to controller 718. A battery 722 supplies electric energy to controller 718 and receiver 720.

Receiver 720 receives electromagnetic signals containing information about a required change in size of the adjustable opening. Receiver 720 converts the electromagnetic signals to electric signals and transmits the electric signals to controller 718. Controller 718 calculates the required electric current to cause the required change in size of the adjustable opening. The required electric current is then delivered to motor 716 causing driver gear 714 to rotate. Rotation of driver gear 714 causes blade actuating ring 708 to rotate. Rotation of blade actuating ring 708 changes orientation of blades 704. This changes the size of lumen 707. Thus, the size of adjustable opening in the invention can be changed. In one embodiment, controller 718, receiver 720 and battery 722 are implanted in the patient's body. The electromagnetic signals are generated out of the patient's body by an external remote controller. Thus, the size of the adjustable opening can be adjusted by a non-invasive procedure. Several biocompatible materials like titanium alloys, stainless steel alloys or elastic biocompatible polymers can be used for constructing the iris diaphragm 700.

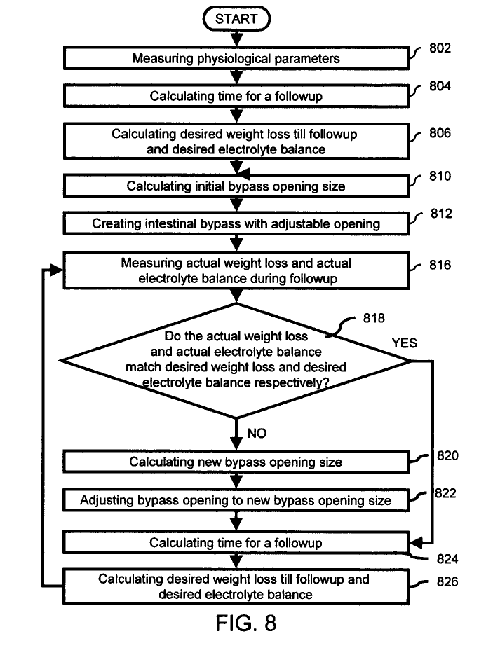

FIG. 8 illustrates the method of the present invention to achieve weight loss in obese patients.

The method of the present invention is based on periodically monitoring the patient's physiological parameters and adjusting the size of the intestinal bypass opening. At step 802, the patient's initial physiological parameters are measured. Some examples of the physiological parameters that are measured are total weight, body mass index, concentration of blood glucose and electrolyte balance. Electrolyte balance is the balance of physiologically crucial compounds like vitamins, and serum electrolytes such as calcium, magnesium, iron and phosphate. Based on these physiological parameters, at step 804, a time is fixed for the followup of the patient. The aim of the followup is to monitor the patient's health status and the effectiveness of the weight loss method. At step 806, a desired weight loss is calculated based on the patient's physiological parameters. The desired weight loss is in the form of a range of weight loss that is desired in the patient until the followup. Also, at step 806, a desired electrolyte balance is calculated for the patient. A proper balance of electrolytes such as calcium, magnesium, iron and phosphate and of vitamin D is crucial for the normal functioning of the body. A poorly designed weight loss program can lead to an excessive loss of electrolytes from the body. At step 810, an initial bypass opening size is calculated based on the patient's physiological parameters, the desired weight loss and the desired electrolyte balance. At step 812, an intestinal bypass with an adjustable opening is surgically created in the patient. The size of the adjustable opening is the initial bypass opening size determined at step 810. Thereafter, the patient is discharged from the hospital and is asked to appear for followup at the time calculated at step 804. During the followup, at step 816, the patient's actual weight loss and actual electrolyte balance is measured. At step 818, the desired weight loss and the actual weight loss are compared. Also, at step 818, the desired electrolyte balance and the actual electrolyte balance are compared. If the desired weight loss and the actual weight loss are not comparable or if the desired electrolyte balance and the actual electrolyte balance are not comparable, the method proceeds to step 820. At step 820, a new bypass opening size is calculated. The calculation is done by taking into consideration the desired weight loss, the actual weight loss, the desired electrolyte balance and the actual electrolyte balance. At step 822, the intestinal bypass is adjusted to the new bypass opening size calculated at step 820. At step 824, a time is fixed for the followup of the patient. At step 826, a desired weight loss is calculated. The desired weight loss is in the form of a range of weight loss that is desired in the patient until the followup calculated at step 824. Also, at step 826, a desired electrolyte balance is calculated for the patient. Thereafter, the method proceeds to step 816.

Referring back to step 818, if at step 818, the desired weight loss and the actual weight loss are comparable and the desired electrolyte balance and the actual electrolyte balance are comparable, the method proceeds to step 824.

While the preferred embodiments of the invention have been described, it will be clear that the invention is not limited to these embodiments only. Several modifications, changes, variations, substitutions and equivalents will be apparent to persons skilled in the art without departing from the spirit and scope of the invention as described in the claims.

Obesity bypass device above mentioned can be coated with drugs such as antibiotics in order to reduce device related infections.

I claim:

1. A device for causing weight loss in obese humans comprising:
an implant that creates an intestinal bypass between a first region of intestine and a second region of intestine.

2. The weight loss device as recited in claim 1, wherein the implant comprises a valve mechanism that allows flow of food material only in one direction.

3. The weight loss device as recited in claim 1, wherein the implant is tubular.

4. The weight loss device as recited in claim 3, wherein the implant comprises an elastic mechanism to facilitate transfer of food material.

5. The weight loss device as recited in claim 3, wherein the implant comprises a series of projections on the inner surface of the implant to facilitate transfer of food material in one direction.

6. The weight loss device as recited in claim 3, wherein the walls of the implant are hollow and are filled with a filler material.

7. The weight loss device as recited in claim 1, wherein the implant comprises a ring that creates a direct physical connection between the first region of intestine and the second region of intestine.

8. The weight loss device as recited in claim 1, wherein the implant is connected to the intestine by biocompatible fasteners selected from the group comprising sutures, clips, staples, screws, tags and adhesives.

9. The weight loss device as recited in claim 1, wherein the implant comprises an adjustable opening to adjust the fraction of food material passing through the intestinal bypass.

10. The weight loss device as recited in claim 9, wherein the size of the adjustable opening can be adjusted by endoscopic means.

11. The weight loss device as recited in claim 9, further comprising a control system for adjusting the size of the adjustable opening; the control system comprising:
a. an external remote controller for transmitting electromagnetic signals, wherein the electromagnetic signals contain information for adjusting the size of the adjustable opening,
b. a receiver for
i. receiving electromagnetic signals from the external remote controller and
ii. converting them to electrical signals,
c. a control mechanism for
i. receiving electrical signals from the receiver and
ii. adjusting the size of the adjustable opening and
d. an energy storage mechanism for supplying energy to the receiver and the control mechanism.

12. A device for causing weight loss in obese humans comprising:
an implant that creates an intestinal bypass between a first region of intestine and a second region of intestine; wherein the implant comprises an adjustable opening to adjust the fraction of food material passing through the intestinal bypass.

13. The weight loss device as recited in claim 12, wherein the implant comprises a valve mechanism that allows flow of food material only in one direction.

14. The weight loss device as recited in claim 12, wherein the implant is tubular.

15. The weight loss device as recited in claim 14, wherein the implant comprises an elastic mechanism to facilitate transfer of food material.

16. The weight loss device as recited in claim 14, wherein the implant comprises a series of projections on the inner surface of the implant to facilitate transfer of food material in one direction.

17. The weight loss device as recited in claim 14, wherein the walls of the implant are hollow and are filled with a filler material.

18. The weight loss device as recited in claim 12, wherein the implant comprises a ring that creates a direct physical connection between the first region of intestine and the second region of intestine.

19. The weight loss device as recited in claim 12, wherein the implant is connected to the intestine by biocompatible fasteners selected from the group comprising sutures, clips, staples, screws, tags and adhesives.

20. The weight loss device as recited in claim 12, wherein the size of the adjustable opening can be adjusted by endoscopic means.

21. The weight loss device as recited in claim 12, further comprising a control system for adjusting the size of the adjustable opening; the control system comprising:
a. an external remote controller for transmitting electromagnetic signals, wherein the electromagnetic signals contain information for adjusting the size of the adjustable opening,
b. a receiver for
i. receiving electromagnetic signals from the external remote controller and
ii. converting them to electrical signals,
c. a control mechanism for
i. receiving electrical signals from the receiver and
ii. adjusting the size of the adjustable opening and
d. an energy storage mechanism for supplying energy to the receiver and the control mechanism.

22. A device for causing weight loss in obese humans comprising:
an implant that creates an intestinal bypass between a first region of intestine and a second region of intestine; wherein the implant comprises:
1. an adjustable opening to adjust the fraction of food material passing through the intestinal bypass and
2. a valve mechanism that allows flow of food material only in one direction.

23. The weight loss device as recited in claim 22, wherein the implant is tubular.

24. The weight loss device as recited in claim 23, wherein the implant comprises an elastic mechanism to facilitate transfer of food material.

25. The weight loss device as recited in claim 23, wherein the implant comprises a series of projections on the inner surface of the implant to facilitate transfer of food material in one direction.

26. The weight loss device as recited in claim 23, wherein the walls of the implant are hollow and are filled with a filler material.

27. The weight loss device as recited in claim 22, wherein the implant comprises a ring that creates a direct physical connection between the first region of intestine and the second region of intestine.

28. The weight loss device as recited in claim 22, wherein the implant is connected to the intestine by biocompatible fasteners selected from the group comprising sutures, clips, staples, screws, tags and adhesives.

29. The weight loss device as recited in claim 22, wherein the size of the adjustable opening can be adjusted by endoscopic means.

30. The weight loss device as recited in claim 22, further comprising a control system for adjusting the size of the adjustable opening; the control system comprising:
a. an external remote controller for transmitting electromagnetic signals, wherein the electromagnetic signals contain information for adjusting the size of the adjustable opening,
b. a receiver for
i. receiving electromagnetic signals from the external remote controller and
ii. converting them to electrical signals,
c. a control mechanism for
i. receiving electrical signals from the receiver and
ii. adjusting the size of the adjustable opening and
d. an energy storage mechanism for supplying energy to the receiver and the control mechanism.

31. A method for causing weight loss in obese humans comprising the steps of:
a. surgically creating an intestinal bypass with an adjustable opening, the intestinal bypass having an initial bypass opening size,
b. calculating a time for a followup,
c. calculating a desired weight loss of the patient till the followup,
d. calculating a desired electrolyte balance of the patient,
e. calculating actual weight loss and actual electrolyte balance during the followup,
f. if the actual weight loss and the actual electrolyte balance match the desired weight loss and the desired electrolyte balance respectively:
i. calculating a time for a next followup,
ii. calculating a desired weight loss of the patient till the next followup, and
iii. calculating a desired electrolyte balance of the patient,
else
i. calculating a new bypass opening size based on the desired weight loss, the actual weight loss, the desired electrolyte balance and the actual electrolyte balance,
ii. changing bypass opening size to the new bypass opening size,
iii. calculating a time for a followup,
iv. calculating a desired weight loss of the patient till the followup, and
v. calculating a desired electrolyte balance of the patient and
g. repeating steps (e) through (f).

32. The method as recited in claim 31, wherein the method is used in conjunction with existing weight loss methods selected from the group comprising diet modification, exercise therapy and pharmacological therapy.

33. Obesity bypass device as recited in claim 1, wherein components are coated with drugs such as antibiotics in order to reduce device related infections.
